SEQUENCE LISTING (2 sequences)

SEQ ID NO: 1 - protein, 406 aa, human
MIHTNLKKKFSCCVLVFLLFAVICVWKEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

SEQ ID NO: 2 - protein, 380 aa, human
KEKKKGSYYDSFKLQTKEFQVLKSLGKLAMGSDSQSVSSSSTQDPHRGRQTLGSLRGLAKAKPEASFQVWNKDSSSKNLIPRLQKIWKNYLSMNKYKVSYKGPGPGIKFSAEALRCHLRDHVNVSMVEVTDFPFNTSEWEGYLPKESIRTKAGPWGRCAVVSSAGSLKSSQLGREIDDHDAVLRFNGAPTANFQQDVGTKTTIRLMNSQLVTTEKRFLKDSLYNEGILIVWDPSVYHSDIPKWYQNPDYNFFNNYKTYRKLHPNQPFYILKPQMPWELWDILQEISPEEIQPNPPSSGMLGIIIMMTLCDQVDIYEFLPSKRKTDVCYYYQKFFDSACTMGAYHPLLYEKNLVKHLNQGTDEDIYLLGKATLPGFRTIHC

(19) United States
(12) Patent Application Publication
(10) Pub. No.: US 2014/0242058 A1
(43) Pub. Date: Aug. 28, 2014

(54) ST6GAL I MEDIATED MODULATION OF HEMATOPOIESIS

(71) Applicants: Joseph Lau, Hamburg, NY (US); Mehrab Nasiri Kenari, Amherst, NY (US)

(72) Inventors: Joseph Lau, Hamburg, NY (US); Mehrab Nasiri Kenari, Amherst, NY (US)

(73) Assignee: Health Research, Inc., Buffalo, NY (US)

(21) Appl. No.: 14/128,642

(22) PCT Filed: Jun. 25, 2012
(86) PCT No.: PCT/US12/43966

§ 371 Date: Apr. 8, 2014

Related U.S. Application Data
(60) Provisional application No. 61/501,093, filed on Jun. 24, 2011.

(51) Int. Cl. A61K 38/45

(52) U.S. Cl. CPC A61K 38/45
USPC 424/94.5

(57) ABSTRACT

Provided are methods for reducing haematocytes in an individual. The method involve administering to an individual a composition that contains recombinant a2,6-sialyltransferase (ST6Gal I). The method is suitable for prophylaxis and therapy of a condition that is positively correlated with undesirable hematopoiesis. Such conditions include autoimmune diseases, transplantation rejection, blood cancers and allergic reactions and inflammations. The invention also provides a pharmaceutical preparation that contains recombinant ST6Gal I and which is suitable for administration to an individual to reduce haematocytes in the individual, and a pharmaceutically acceptable carrier.

This application claims priority to U.S. provisional application No. 61/501,093, filed Jun. 24, 2011, the disclosure of which is incorporated herein by reference.

This invention was made with government support under grant no. AI-056082 awareded by the National Institutes of Health. The government has certain rights in the invention.

### FIELD OF THE INVENTION

The present invention relates generally to modulating hematopoiesis and more specifically to methods for prophylaxis and/or therapy of conditions correlated with undesirable hematopoietic processes.

### BACKGROUND OF THE INVENTION

Hematopoiesis is the mechanism that produces circulating blood cells and certain other cells that participate in immune responses in various tissues. Many disease conditions that affect vast numbers of people involve aberrant activity of such cells, including various cancers, autoimmune disorders, organ and tissue transplantation rejections, and multiple conditions that involve undesirable inflammation as a component of disease etiology. However, a lack of effective methods for modulation of hematopoiesis for prophylactic and/or therapeutic benefit has been a longstanding problem in medicine. The present invention meets these and other needs.

### SUMMARY OF THE INVENTION

The present invention provides a method for reducing haematocytes in an individual. The method comprises administering to the individual a composition comprising recombinant a2,6-sialyltransferase (ST6Gal I), wherein the administration results in a reduction of haematocytes in the individual. The method is broadly applicable to prophylaxis and/or therapy of a condition that is positively correlated with undesirable hematopoiesis, such as autoimmune diseases, transplantation rejection, certain types of cancers such as leukemias, lymphomas and myelomas, inflammation, allergic reactions, and a variety of other conditions that involve the activity of blood cells. Thus, in various embodiments, and individual who can benefit from the invention include individuals who are diagnosed with or at risk for developing a blood cancer, or those who are candidates for or are recipients of cell, tissue or organ transplantations, or have allergic conditions.

In various embodiments, the haematocytes that are reduced in the individual comprise leukocytes. In an embodiment, the leukocytes comprise granulocytes, or circulating lymphocytes. The haematocytes can also comprise circulating platelets. A reduction in haematocytes can include a reduction in bone marrow cellularity of the individual. The invention also provides a pharmaceutical preparation suitable for administration to an individual to reduce haematocytes in the individual. The pharmaceutical preparation comprises recombinant ST6Gal I and a pharmaceutically acceptable carrier.

### BRIEF DESCRIPTION OF THE FIGURES

FIG. 1 provides a graphical representation of data showing ST6Gal-1 profiles of animals undergoing OVA or ABPA models of allergic pulmonary inflammation.

FIG. 2 provides a graphical representation of data showing greater neutrophilia in SiatlΔP1 and Siatl-null animals.

FIG. 3 provides a graphical representation of data showing suppression of G-CSF elicited release of white cells, including neutrophils, by recombinant ST6Gal I infusion i.v.

FIG. 4 provides a graphical representation of data showing LSK (Lin-: Sca+:cKit+) cells from SiatlΔP1 mice have greater proliferation in vitro.

FIG. 5 provides a graphical representation of data showing supplementation of in vitro cultures of C57BL/6 bone marrow cells with recombinant ST6Gal I (rST6G) resulted in depressed IL-3/G-CSF dependent and IL-5 dependent colonies.

FIG. 6 provides a graphical representation of data showing SiatlΔP1 donors and recipients are less able to retain transfused donor stem cells.

FIG. 7 provides a graphical representation of data showing elevated eosinophil infiltration in the bronchoalveolar lavage of ST6Gal-1-deficient mice upon allergen provocation.

FIG. 8 provides a graphical representation of data showing allergic airway inflammation is attenuated by bolstering systemic ST6Gal-1.

FIG. 9 provides a graphical representation of data showing loss of 33% of total nucleated cell numbers in bone marrow after administration of recombinant ST6Gal 1.

FIG. 10 provides a graphical representation of data showing flow analysis of bone marrow nucleated cells for CDllb and Ly6G showing depletion of granulocyte reservoir in marrow (circled population) after administration of recombinant ST6Gal 1.

FIG. 11 provides a graphical representation of data showing a decrease in total white cell counts (WBC), lymphocytes (LYMPH), and platelets (PLT) in circulation after administration of recombinant ST6Gall.

### DESCRIPTION OF THE INVENTION

The present invention provides compositions and methods for modulating hematopoiesis to achieve a prophylactic and/or therapeutic benefit in an individual. The method comprises administering a composition comprising recombinant ST6Gal Ito an individual. The administration results in prophylaxis and/or therapy of a condition that is positively correlated with undesirable hematopoiesis. Thus, in various embodiments of the invention, methods are provided for administering a composition comprising an effective amount of recombinant ST6Gal Ito an individual in need of treatment for a condition for which modulation of hematopoiesis would be desirable.

ST6Gal I (also referred to as ST6GalI and ST6Gal1 ST6Gal-I) is a sialyltransferase that constructs the sialyl a2,6 to Gal β1,4GlcNAc glycan structure common on many cell surface and circulatory glycoproteins. Transcription of the ST6Gal I gene is mediated by 6 physically distinct promoter/transcriptional initiation regions. In the native form, ST6Gal I is localized in the Golgi, where it participates in the assembly of sialyl-glycoconjugates transiting the secretory apparatus. The intact catalytic domain can be proteolytically liberated and released into systemic circulation as the soluble ST6Gal I form. Therefore, ST6Gal I can be divided into two conceptual categories: The “cell-restricted” ST6Gal-1 that remains within the cells that produced them, and the “circulatory”, or “soluble”, ST6Gal-1 that has been released into systemic circulation. Circulatory ST6Gal I originates predominantly from the liver; specific inactivation of the liver-restricted promoter (P1) of the ST6Gal I gene results in depressed systemic ST6Gal I levels.

Liver synthesized ST6Gal I either remains in a cell-restricted manner and participates in sialylation of liver-derived circulatory glycoproteins, or it can be released into circulation as circulatory/systemic ST6Gal I. Because inactivation of P1 results in negligible alteration to the sialylation of liver-derived serum glycoproteins, the principal and immediate biosynthetic consequence of P1 inactivation is the suppression of systemic ST6Gal I levels. However, there has been no previous recognition or demonstration that exogenously supplied ST6Gal I can affect hematopoiesis in vivo, especially for the purpose of providing a prophylactic and/or therapeutic effect.

The present invention is based in part on our discovery that systemic ST6Gal I, levels of which can be increased by administration of recombinant ST6Gal I, provides a novel axis to modulate hematopoietic homeostatic balance and, among other effects, to control production of all blood cells, including platelets, lymphocytes and inflammatory cells and their release into circulation. We demonstrate that systemic ST6Gal I level is decreased during acute inflammation or during increased myelopoietic activity; G-CSF mediated release of granulocytes from the bone marrow is enhanced in systemic ST6Gal I deficient mice; G-CSF mediated release of granulocytes is suppressed by i.v. infusion of recombinant ST6Gal I; hematopoietic stem/progenitor cell proliferation and differentiation is elevated in systemic ST6Gal I deficient mice; differentiation and proliferation is suppressed in the presence of recombinant ST6Gal I; ST6Gal I deficient animals are less able to retain transfused donor stem cells; acute allergic airway inflammation is more severe in ST6Gal I deficient animals; pulmonary inflammatory cell numbers are strikingly attenuated by increasing systemic ST6Gal I by adenoviral-mediated therapy. In addition to these observations, and importantly, we also demonstrate that administration of a composition comprising recombinant ST6Gal I in vivo results in a reduction in total bone marrow cellularity, including a depletion of granulocyte reservoir in marrow, as well as a reduction of total circulating leukocytes and platelets, as well as total circulating lymphocytes.

Without intending to be constrained by any particular theory, it is considered that in various embodiments, the invention facilitates a broad suppression of hematopoiesis such that formation of cells of a hematopoietic origin in an individual is inhibited. In particular, the invention provides for lowering the amount of haematocytes in an individual. Those skilled in the art will recognize that suppression of hematopoiesis provided by the invention is reversible, being dependent on the presence and amount of exogenously supplied ST6Gal I.

Suppression of hematopoiesis according to the method of the invention can be evidenced in a variety of ways. For example, in various embodiments, the invention results in a lowering of the amount of haematocytes, wherein the haematocytes are present in the circulatory system, the lymphatic system, the marrow, or in combinations thereof. In certain embodiments, the invention results in a lowering of the amount of erythrocytes, thrombocytes, leukocytes, or combinations thereof in the individual. In one embodiment, lowering the amount of leukocytes comprises lowering the amount of granulocytes in an individual. Lowering the amount of granulocytes in certain embodiments comprises lowering the amounts of neutrophils, eosinophils, basophils, or combinations thereof. In another embodiment, lowering the amount of leukocytes in an individual comprises lowering the amount of agranulocytes in the individual. The agranulocytes, production of which is affected by the invention, can include lymphocytes, monocytes, macrophages, and combinations thereof. The lymphocytes can include B-cells, T-cells, natural killer (NK) cells, and combinations thereof.

In certain aspects of the invention, and again without intending to be limited by any particular theory, it is considered that lowering the amount of cells of hematopoietic origin in an individual is effectuated by modulating hematopoietic stem/progenitor cells. Thus, the invention can affect multipotential hematopoietic stem cells (hemocytoblasts), which in turn can differentiate into either common myeloid progenitor cells or common lymphoid progenitor cells. In connection with common myeloid progenitor cells, it is expected that the invention can affect their differentiation pathway from megakaryoblasts to thrombocyte formation, and/or the differentiation pathway of myeloblasts to basophils, neutrophils and eosinophils, and/or to monocytes, e.g., macrophages or myeloid dendritic cells. In connection with common lymphoid progenitor cells, it is expected that the invention can affect their differentiation into lymphoblasts, thereby inhibiting production of B lymphocytes and plasma cells, T lymphocytes, NK cells, and lymphoid dendritic cells. In particular aspects the invention facilitates lowering total bone marrow cellularity, e.g., depletion of total nucleated cell numbers in bone marrow, and/or depleting granulocyte reservoir in marrow.

In various embodiments, the hematopoietic inhibitory effects facilitated by administering a composition comprising ST6Gal Ito an individual according to the invention results in prophylaxis and/or therapy for a condition wherein a dampened immune response is desirable. Thus, the invention comprises a method for prophylaxis and/or therapy of myeloproliferative disorders, which include but are not necessarily limited to Polycythemia vera, Primary or idiopathic myelofibrosis (myelosclerosis), Essential thrombocytosis, and blood cancers, such as a myeloma, including multiple myeloma, or a lymphoma, or a leukemia, and particularly chronic myelogenous leukemia (CML).

The invention also provides for prophylaxis and/or therapy of conditions that are characterized by undesirable inflammation. Examples of such conditions include but are not necessarily limited to chronic inflammatory diseases, which include but are not necessarily limited to chronic obstructive pulmonary disease (COPD), irritable bowel syndrome, and atherosclerosis.

The invention also provides for prophylaxis and/or therapy of conditions that are characterized by allergic reactions. Thus, in various embodiments, the invention is useful for lessening the severity of, for instance, type I hypersensitivity reactions and/or late phase allergic responses. Non-limiting examples of allergic reactions for which the present invention can provide a prophylactic and/or therapeutic benefit include allergic rhinitis, food allergies, asthma and related airway inflammatory conditions, allergic reactions caused by envenomation or medications.

The invention also provides for prophylaxis and/or therapy of autoimmune diseases characterized by an inappropriate immune response against self-antigens or other substances that are normally present in the body. Non-limiting examples of autoimmune diseases for which the present invention can provide a prophylactic and/or therapeutic benefit include those which are characterized by type II, III or IV hypersensitivity. Particular, non-limiting examples include celiac disease, Crohn's disease, diabetes mellitus type 1, eosinophilic fasciitis, eosinophilic gastroenteritis, gastritis, Graves' disease, hypogammaglobulinemia, idiopathic inflammatory demyelinating diseases, thrombocytopenic purpura, rheumatoid arthritis, lupus erythematosus, myasthenia gravis, pernicious anaemia, psoriasis, Sjogren's syndrome, and ulcerative colitis.

The invention is also suited for use in connection with transplantations, such as allogeneic and autologous bone marrow transplantations, stem cell transplantation, adoptive T cell therapies, and tissue and organ transplantations, such as for prophylaxis and/or therapy of transplant rejection processes, including but not limited to graft versus host disease. Thus, in various embodiments, the individual to whom a composition comprising recombinant ST6Gal I is administered according to the invention is a candidate for, or is a recipient of a cell, tissue or organ transplantation.

Each of the cell types of hematopoietic origin can be lowered in individual relative to the amount of the cell type prior to administration of a composition of the invention to the individual. The lowered amounts of the cells can occur in marrow, lymph, the cardiovascular system, the lymphatic system, lymphatic tissue, tissue which has become inflamed, or combinations thereof. The degree of lowering the cell population can be any desirable degree. In various embodiments, the amount of cells can be lowered by 1% -100%, inclusive, and including all integers there between.

For practicing the method of the invention, recombinant ST6Gal I can be isolated or synthesized using any suitable techniques, and commercially produced recombinant ST6Gal I is available from, for example, Novoprotein, Short Hills, N.J.

The amino acid sequence of human ST6Gal I proteins are known in the art. For reference, ST6Gal I amino acid sequences are as follows:

Complete sequence (as synthesized and as exist in the “cell-restricted” form as a 406aa protein (SEQ ID NO:1):

| | |
|---|---|
| 1 | mihtnlkkkf sccvlvfllf avicvwkekk kgsyydsfkl qtkefqvlks lgklamgsds |
| 61 | qsvsssstqd phrgrqtlgs lrglakakpe asfqvwnkds ssknliprlq kiwknylsmn |
| 121 | kykvsykgpg pgikfsaeal rchlrdhvnv smvevtdfpf ntsewegylp kesirtkagp |
| 181 | wgrcavvssa gslkssqlgr eiddhdavlr fngaptanfq qdvgtkttir lmnsqlvtte |
| 241 | krflkdslyn egilivwdps vyhsdipkwy qnpdynffnn yktyrklhpn qpfyilkpqm |
| 301 | pwelwdilqe ispeeiqpnp pssgmlgiii mmtlcdqvdi yeflpskrkt dvcyyyqkff |
| 361 | dsactmgayh pllyeknlvk hlnqgtdedi yllgkatlpg frtihc |

Circulatory/systemic form 380aa protein (generated by proteolytic cleavage of parental “cell-restricted” form). Due to ambiguity of proteolytic action, the first 4-8 AA residues may or may not be present. Recombinant proteins used in the method of the invention may accordingly lack the first 4, 5, 6, 7 or 8 amino acids shown in the following sequence (SEQ ID NO:2)

kekk kgsyydsfkl qtkefqvlks lgklamgsds qsvsssstqd phrgrqtlgs lrglakakpe
asfqvwnkds ssknliprlq kiwknylsmn kykvsykgpg pgikfsaeal rchlrdhvnv
smvevtdfpf ntsewegylp kesirtkagp wgrcavvssa gslkssqlgr eiddhdavlr
fngaptanfq qdvgtkttir lmnsqlvtte krflkdslyn egilivwdps vyhsdipkwy
qnpdynffnn yktyrklhpn qpfyilkpqm pwelwdilqe ispeeiqpnp pssgmlgiii
mmtlcdqvdi yeflpskrkt dvcyyyqkff dsactmgayh pllyeknlvk hlnqgtdedi
yllgkatlpg frtihc

Additional information about the ST6Gal I sequence can be found in NCBI accession no. P15907 (Apr 1, 1990 entry), which is incorporated herein by reference as it exists on the priority date for this disclosure.

The invention includes using recombinant ST6Gal I (also referred to as “rST6Gal I” or “rST6G”) that is identical to the known human sequences shown above, or polypeptides that have an amino acid sequence that has greater than about 70% amino acid sequence identity, preferably about 75, 80, 85, 90, or 95% or more amino acid sequence identity, to the known sequence of human ST6Gal I.

The rST6Gal I used in the invention may have conservative substitutions which are based generally on relative similarity of R-group substituents. As examples, these substitutions include gly or ser for als; lys for arg; gln or his for asn; glu for asp; ser for cys; asn for gln; asp for glu; ala for gly; asn or gln for his; leu or val for ile; ile or val for leu; arg for lys; leu or tyr for met; thr for ser; tyr for trp; phe for tyr; and ile or leu for val.

For use in the methods of the invention, a composition comprising rST6Gal I can be prepared as therapeutic formulations by mixing rST6Gal I with any suitable pharmaceutically acceptable carriers, excipients and/or stabilizers. Some examples of compositions suitable for mixing with the agent can be found in: Remington: The Science and Practice of Pharmacy (2005) 21st Edition, Philadelphia, Pa. Lippincott Williams & Wilkins. Thus, in various embodiments, the invention provides a pharmaceutical preparation comprising rST6Gal I.

The compositions of the invention can be administered using any suitable method and route of administration. Some non-limiting examples include oral, parenteral, subcutaneous, intraperitoneal, intrapulmonary, and intranasal. Parenteral infusions include intramuscular, intravenous, intraarterial, intraperitoneal, pulmonary instillation as mist or nebulization, and subcutaneous administration.

Administration of the compositions of the invention can be performed in conjunction with conventional therapies that are intended to treat a disease or disorder, wherein the conventional therapies entail or would benefit from modulation of hematopoietic homeostatic balance and/or controlling production of inflammatory cells, their release into circulation and accumulation in inflammatory sites.

For example, the composition could be administered prior to, concurrently, or subsequent to conventional anti-cancer therapies. Such therapies include but are not limited to chemotherapies, surgical interventions, radiation therapy, and other treatment modalities that relate to blood cancers or therapies wherein a reduction of inflammation is desired.

In general, an appropriate dosage and treatment regimen provides the composition in an amount effective to modulate hematopoietic homeostatic balance and/or control production of inflammatory cells and their release into circulation and availability to a desired degree. A desired response can be monitored by an improved clinical outcome according to parameters that will be apparent to those skilled in the art, dependant upon the condition being treated.

Routes and frequency of administration of the therapeutic compositions disclosed herein, as well as dosage, will vary from individual to individual, and may be readily established using standard techniques given the benefit of the present disclosure. Those skilled in the art will recognize how to formulate for pharmaceutical preparations comprising rST6Gal I, and appropriate dosing can be determined by taking into account such factors as the size, age, gender and health of the individual to be treated, and the type and stage of disease or condition. The compositions comprising rST6Gal I can be administered prior to, concurrently, or subsequent to administration of other agents or the performance of any other medical protocol that is desirable for treating the individual.

The following examples are intended to illustrate, but not limit the invention.

### EXAMPLE 1

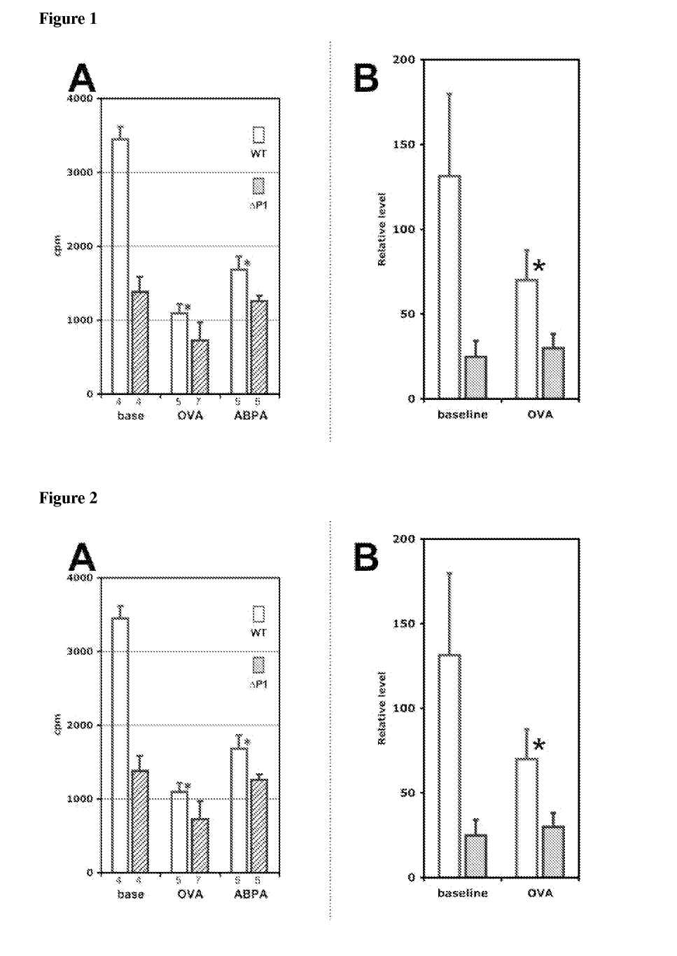

As will be evident from FIG. 1, this Example demonstrates that systemic ST6Gal I level is decreased during acute inflammation or during increased myelopoietic activity. In FIG. 1, panel A, serum sialyltransferase activity profiles are shown. Serum was harvested from wild-type (WT) and SiatlΔP1 (ΔP1) mice either at rest (base) or undergoing OVA or ABPA protocols of allergic airway inflammation and tested for sialyltransferase activity. Shown is the 3[H] incorporation into the synthetic acceptor substrate, GalNAc(β1,4)GlcNAc-o-Bz from 30 Ci/mmol CMP-3[H]NeuNAc by 10 μl serum after 2 hr incubation at 37° C. The numbers immediately beneath the abscissa indicate the number of animals comprising each data bar. Statistical significance is reached (p<0.01) for WT undergoing either OVA or ABPA when compared to baseline ( [symbol] ), and also for difference between WT and ΔP1 upon OVA provocation. Panel B, real time RT-PCR analysis of liver ST6Gal-1 mRNA if WT and ΔP1 mice either at rest (baseline) or undergoing the OVA protocol. Open bar is represents the wild-type (N=3) and hatched bar represents the SiatlΔP1 (ΔP1) mice (N=3). Statistical significance was reached (p<0.05) for wild-type upon OVA provocation compared to baseline.

### EXAMPLE 2

This Example demonstrates greater neutrophilia in SiatlΔP1 and Siatl-null animals. As will be evident from FIG. 2, G-CSF mediated release of granulocytes from bone marrow is enhanced in systemic ST6Gal I deficient mice. To obtain the data summarized in FIG. 2, peripheral blood was collected in mice in the absence of treatment (resting, left panel) or 30 minutes after administration of G-CSF i.v. (middle and right panels). The collected blood was analyzed by flow cytometry after lysis of the red blood cells. The granulocyte population, which are Gr-1 positive, was calculated by taking the percentage of the Gr-1 positive cells against total events for each flow acquisition. * marks mutant animal data point that was statistically different from wild-type animals by T test (p<0.05).

### EXAMPLE 3

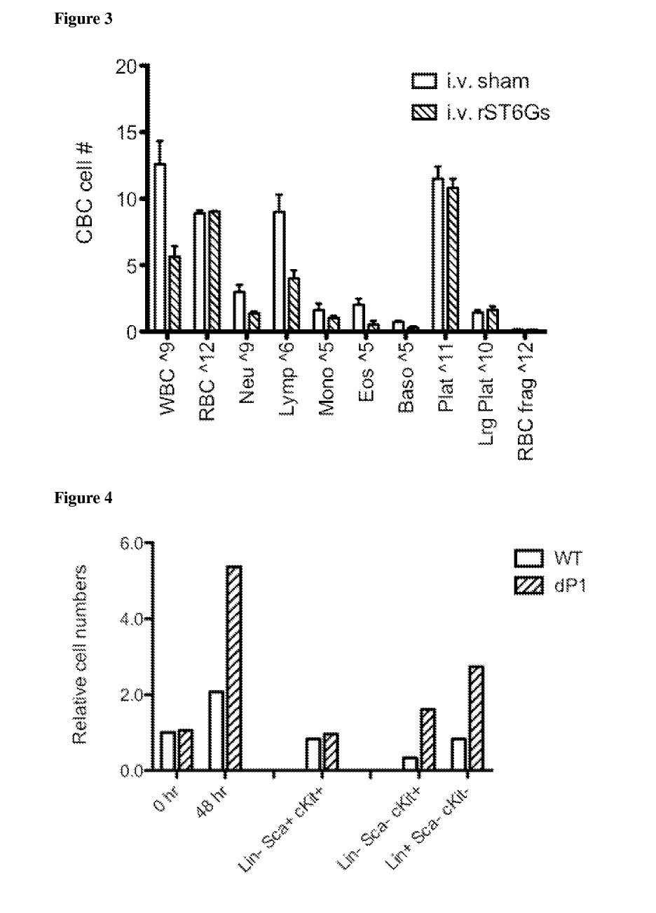

It will be evident from FIG. 3 that G-CSF mediated release of granulocytes is suppressed by i.v. infusion of recombinant ST6Gal I. To obtain the data presented in FIG. 3, recombinant ST6Gal I or PBS vehicle (sham) was infused into recipient C57BL/6 wild-type mice. 24 hours after infusion, blood was withdrawn and subjected to CBC analysis. WBC: total white blood cells, RBC: red blood cells; Neu: neutrophil; Lymph: lymphocytes; Mono: monocytes; Eos: eosinophils; Baso: basophils; Plat: platelets. Differential counts between rST6G and sham injected groups were statistically significant in WBC, Neu, Lymph, and Eos categories.

### EXAMPLE 4

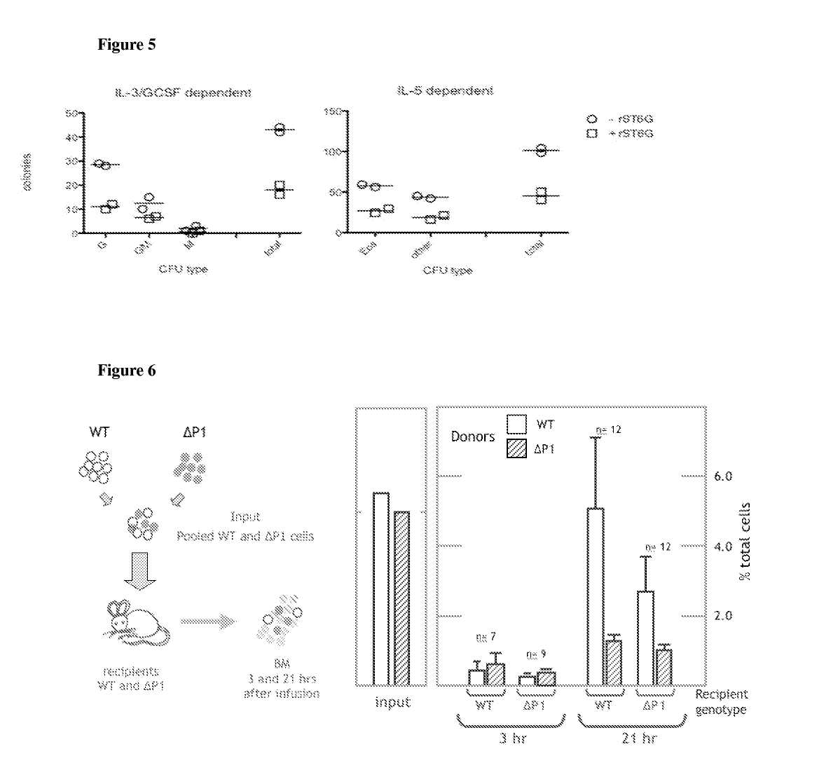

FIG. 4 and FIG. 5 demonstrate that hematopoietic stem/progenitor cell proliferation and differentiation is elevated in systemic ST6Gal I deficient mice, but differentiation and proliferation is suppressed in the presence of recombinant ST6Gal I. In particular, data summarized in FIG. 4 show that LSK (Lin-: Sca+:cKit+) cells from SiatlΔP1 mice have greater proliferation in vitro. To obtain the data for FIG. 4, LSK cells isolated from C57BL/6 wild-type and from SiatlΔP1 anmals were cultured in vitro for 48 hours, and the total cells renumerated. The results showed that, starting from the same number of LSK cells of either genotype, SiatlΔP1 proliferated 2.8-fold greater with greater proportion of cells differentiating into Sca-neg and Lin-pos populations after 48 hrs. Data summarized in FIG. 5 show that supplementation of in vitro cultures of C57BL/6 bone marrow cells with recombinant ST6Gal I (rST6G) resulted in depressed IL-3/G-CSF dependent and IL-5 dependent colonies. To obtain the data summarized in FIG. 5, bone marrow cells from wild-type animals were seeded into semi-solid media in the presence of either IL3 and G-CSF (left panel) or IL-5 (right panel), either with or without addition of rST6G. Seven days later, the cultures were assessed for total numbers (total) of colonies as well as the CFU type. 2- to 3-fold suppression of colony numbers by the presence of recombinant ST6Gal I was observed for G-, GM-, and Eos-CFU's.

### EXAMPLE 5

As will be evident from FIG. 6, ST6Gal I deficient animals are less able to retain transfused donor stem cells. In particular, C57BL/6 wild-type and SiatlΔP1 donor cells were respectively labeled with different fluorescent dyes, mixed in 1:1 ratio and infused into either wild-type or SiatlΔP1 recipients by tail vein injection. The bone marrow of the recipients were harvested 3 and 21 hrs after transfusion, and enumerated for the number of the respective donor cells. WT and SiatlΔP1 cells homed with roughly equal efficiency, but the homing efficiency was significantly diminished in SiatlΔP1 recipients (3 hr point). Stem cell retention was monitored in the 21 hr point, and significantly less SiatlΔP1 cells were retained than wild-type cells. Moreover, the SiatlΔP1 recipients had significantly less retained donor cells of either genotype than wild-type recipients.

### EXAMPLE 6

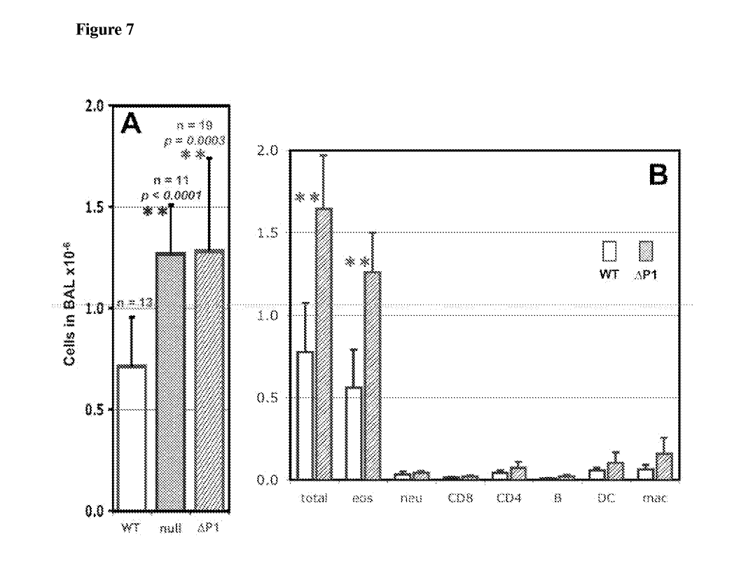

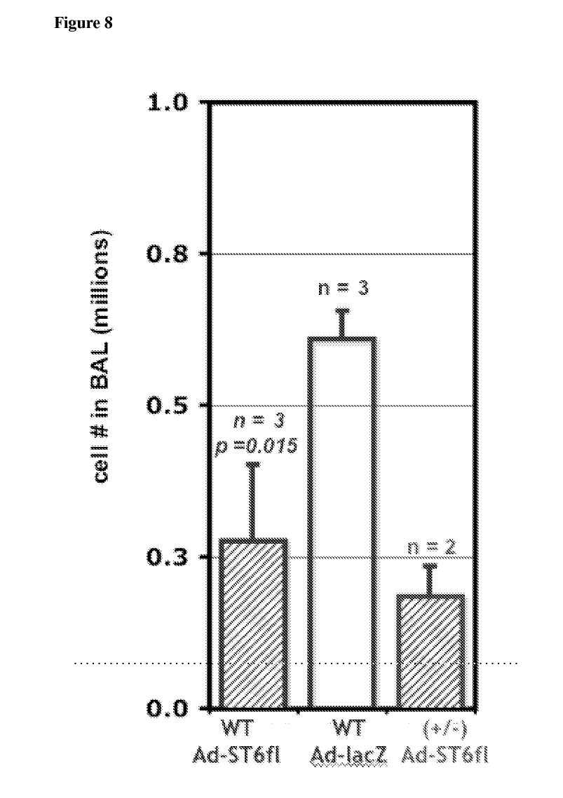

This Example shows that acute allergic airway inflammation is more severe in ST6Gal I deficient animals and that pulmonary inflammatory cell numbers are strikingly attenuated by increasing systemic ST6Gal I by adenoviral-mediated therapy. In particular, FIG. 7 shows elevated eosinophil infiltration in the bronchoalveolar lavage of ST6Gal-1-deficient mice upon allergen provocation. To obtain the data summarized in FIG. 7, bronchoalveolar lavage (BAL) was recovered from wild-type C57BL/6 (WT), SiatlΔP1 (ΔP1), or Siatl-null (Null) animals undergoing acute OVA-provoked allergic pulmonary inflammation. Panel A shows the total cell content, expressed as BAL cells/animal, as determined on a coulter counter. N is the number of animals used for each respective data point. Panel B shows the BAL cell composition of WT (open bars) and SiatlΔP1 (hatched bars) as determined by FACS analysis. The data shown is the mean of 4-5 WT and 4-6 SiatlΔP1 animals, and [symbol] *denotes statistical significance of p<0.003). FIG. 8 demonstrates that allergic airway inflammation is attenuated by bolstering systemic ST6Gal-1. WT (+/+) or mice heterozygous for the Siatl-null mutation (+/−) were inoculated with either Ad-ST6fl or Ad-lacZ (i.v. 108 particles/animal) 5 days prior to OVA challenge. “n” is the number of mice for each respective data point, and “p” is the statistical difference between WT mice receiving Ad-lacZ or Ad-ST6fl.

### EXAMPLE 7

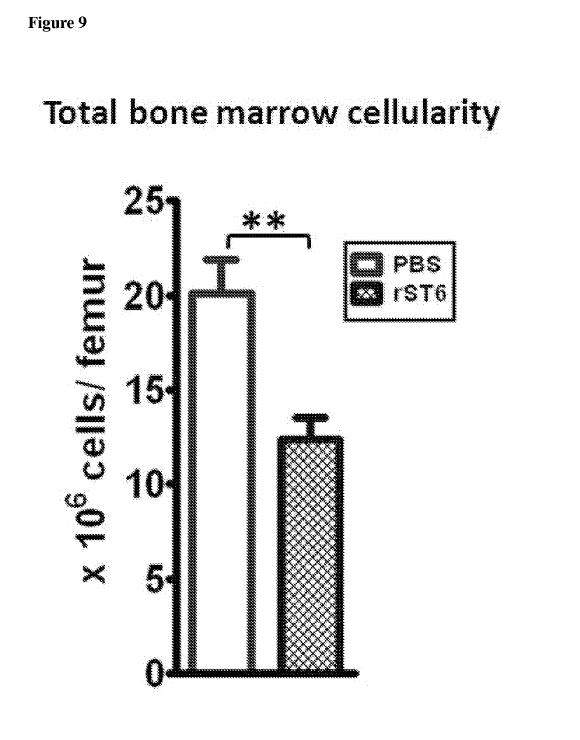

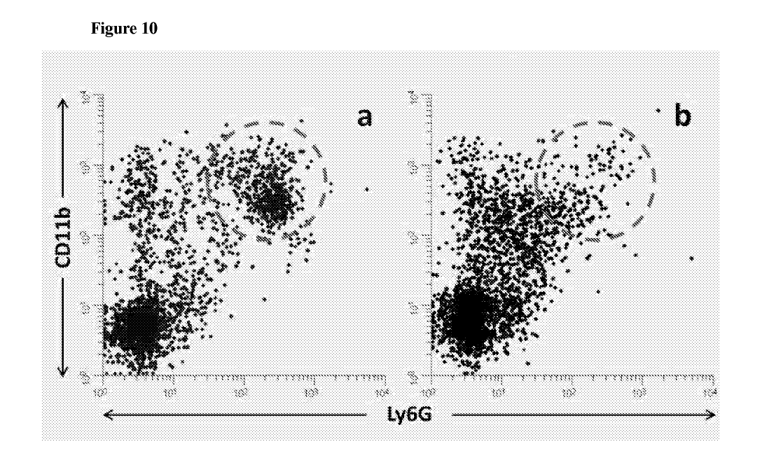

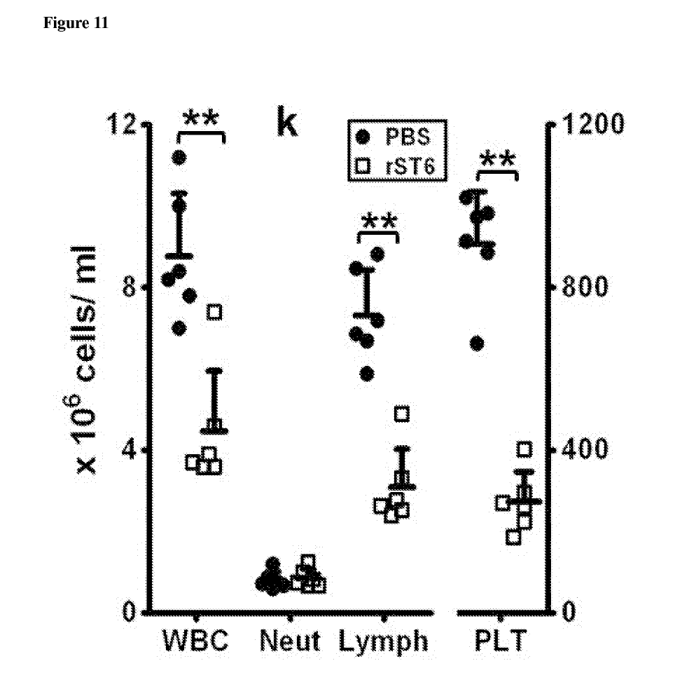

This Example demonstrates that administration of exogenous rST6G I effectuates a reduction in bone marrow cellularity as depicted in FIG. 9. Further, the data depicted in FIG. 10 (flow analysis of bone marrow nucleated cells for CDllb and Ly6G) demonstrates depletion of granulocyte reservoir in marrow (circled population). Further still, FIG. 11 demonstrates that the administration of exogenous rST6G I results in a decrease of total white cell counts (WBC), lymphocytes (LYMPH), and platelets (PLT) in circulation. To obtain the data presented in FIGS. 8, 9 and 10, animals (C57BL/6, males) received 200 ul rST6G (10 mg/Kg rST6G), or saline (PBS), by intravenous injections (3 sequential injections, 8 hours apart). Eight hours after the last injection, circulatory blood counts (CBC) were determined. Nucleated bone marrow cells were extracted from the femurs and counted using TC10 Automated Cell Counter (Bio-Rad). Flow cytometry was performed using APC anti-mouse Ly6G antibody, FITC anti-mouse CDllb antibody and PE anti-mouse CD45R/B220 antibody. This experiment was performed with 6 PBS and 6 rST6G treated mice. The recombinant ST6Gal-1 (rST6G) was the recombinant human catalytic domain was captured as part of the Glycoenzyme repository in pDONR221.

It will be apparent to those skilled in the art from the foregoing description and Examples that the present invention provides a method that is suitable for broad suppression of hematopoiesis in an individual, and it follows that such suppression would be suitable for prophylaxis and/or therapy of conditions that are positively correlated with undesirable hematopoietic processes.

We claim:

1. A method for reducing haematocytes in an individual comprising administering to the individual a composition comprising recombinant a2,6-sialyltransferase (ST6Gal I), wherein the administration results in a reduction of haematocytes in the individual.

2. The method of claim 1, wherein the individual is in need of prophylaxis and/or therapy of a condition that is positively correlated with undesirable hematopoiesis.

3. The method of claim 1, wherein the individual is at risk for, is suspected of having or has been diagnosed as having a condition selected from a blood cancer, an autoimmune disease, a chronic inflammation, an allergic condition, and combinations thereof.

4. The method of claim 3, wherein the blood cancer is selected from leukemia, lymphoma, myeloma, and combinations thereof.

5. The method of claim 3, wherein the autoimmune disease is selected from celiac disease, Crohn's disease, diabetes mellitus type 1, eosinophilic fasciitis, eosinophilic gastroenteritis, gastritis, Graves' disease, hypogammaglobulinemia, idiopathic inflammatory demyelinating diseases, thrombocytopenic purpura, rheumatoid arthritis, lupus erythematosus, myasthenia gravis, pernicious anaemia, psoriasis, Sjogren's syndrome, ulcerative colitis, and combinations thereof.

6. The method of claim 1, wherein the individual is a candidate for, or is a recipient of a cell, tissue or organ transplantation.

7. The method of claim 6, wherein the individual is at risk for developing or has graft versus host disease.

8. The method of claim 1, wherein the haematocytes that are reduced in the individual comprise leukocytes.

9. The method of claim 8, wherein the leukocytes comprise granulocytes.

10. The method of claim 1, wherein the reduction of haematocytes in the individual comprises a reduction in bone marrow cellularity of the individual.

11. The method of claim 10, wherein the reduction in bone marrow cellularity comprises a depletion of granulocytes in the bone marrow of the individual.

12. The method of claim 1, wherein the reduction of haematocytes in the individual comprises a reduction of circulating total white blood cell counts, circulating lymphocytes, circulating platelets, or a combination thereof.

13. The method of claim 12, wherein the reduction of haematocytes in the individual comprises a reduction of circulating total white blood cell counts.

14. The method of claim 12, wherein the reduction of haematocytes in the individual comprises a reduction of circulating lymphocytes.

15. A pharmaceutical preparation comprising recombinant a2,6-sialyltransferase (ST6Gal I) suitable for administration to an individual to reduce haematocytes in the individual, and a pharmaceutically acceptable carrier.
